(19) United States
(12) Patent Application Publication
(10) Pub. No.: US 2015/0309042 A1
(43) Pub. Date: Oct. 29, 2015

(54) FLUOROGENIC DENDRIMER REPORTERS AND RELATED METHODS OF USE

(71) Applicant: The Regents of the University of Michigan, Ann Arbor, MI (US)

(72) Inventors: James R. Baker, JR., Ann Arbor, MI (US); Hong Zong, Ann Arbor, MI (US); Sascha Goonewardena, Ann Arbor, MI (US)

(21) Appl. No.: 14/758,735

(22) PCT Filed: Jan. 6, 2014
(86) PCT No.: PCT/US2014/010337

§ 371 Date: Jun. 30, 2015

Related U.S. Application Data
(60) Provisional application No. 61/752,299, filed on Jan. 14, 2013.

(51) Int. Cl. G01N 33/58; C08G 69/48

(52) U.S. Cl. CPC G01N 33/582; C08G 69/48

(57) ABSTRACT

The present invention relates to fluorogenic dendrimer reporters. In particular, the present invention relates to dendrimer nanoparticles conjugated with ‘click-on’ fluorogenic reporters and related methods of use.

### STATEMENT REGARDING FEDERALLY SPONSORED RESEARCH OR DEVELOPMENT

This invention was made with government support under W911NF-07-1-0437 awarded by the U.S. Army Research Office. The government has certain rights in the invention.

### FIELD OF THE INVENTION

The present invention relates to fluorogenic dendrimer reporters. In particular, the present invention relates to dendrimer nanoparticles conjugated with ‘click-on’ fluorogenic reporters and related methods of use.

### BACKGROUND OF THE INVENTION

Understanding dynamic cellular processes requires the ability to track biomacromolecules and small molecule metabolites in complex biological environments including cells and animal models. Traditionally, molecules have been tracked using genetically encoded fluorescent labels like GFP or with exogenous antibody reporters (see, e.g., Kulkarni, G.; Wadsworth, W. G. Nat Methods 2012, 9, 451; Prescher, J. A.: Contag, C. H. Curr Opin Chem Biol 2010, 14, 80; Hoffman, R. M. Method Enzymol 2012, 506, 197). While these methodologies have greatly enhanced the ability to probe biological systems, these approaches require the creation of bulky fusion proteins and antibody reporters that often perturb cellular processes or have inherent limitations in their efficiency (see, e.g., Prescher, J. A.; Bertozzi, C. R. Nat Chem Biol 2005, 1, 13).

Improved compositions and methods for tracking biomacromolecules and small molecule metabolites in complex biological environments are needed.

### SUMMARY OF THE INVENTION

The application of small molecule fluorescent reporters to monitor biological systems is limited by their poor water solubility and background fluorescence of these reporters. To address such limitations, the present invention provides dendrimer nanoparticles conjugated with a ‘click-on’ fluorogenic reporter (e.g., 3-azido coumarin, 9-azido anthracene, 6-azido napthalimide, 7-ethynyl coumarin, 2-ethynyl benzothiazole, and 6-ethynyl napthalimide) thereby generating fluorogenic dendrimer reporters. Such fluorogenic dendrimer reporters are not limited to particular uses. In some embodiments, such fluorogenic dendrimer reporters (e.g., dendrimers conjugated with a ‘click-on’ fluorogenic reporter (e.g., 3-azido coumarin, 9-azido anthracene, 6-azido napthalimide, 7-ethynyl coumarin, 2-ethynyl benzothiazole, and 6-ethynyl napthalimide)) are used to track functionalized small molecules configured to interact with the ‘click-on’ fluorogenic reporter to develop a triazole ring, thereby permitting the ‘click on’ fluorogenic reporter to fluoresce. As such, in embodiments wherein the ‘click-on’ fluorogenic reporter is azido based (e.g., 3-azido coumarin, 9-azido anthracene, 6-azido napthalimide), the functionalized small molecule is an alkyne functionalized small molecule (e.g., wherein the azido interacts with the alkyne to develop a triazole ring thereby permitting the ‘click-on’ fluorogenic reporter to fluoresce). In some embodiments, the alkyne within the alkyne functionalized small molecule is an alkyne group. In some embodiments, the alkyne within the alkyne functionalized small molecule is a cycloalkyne group. Similarly, in embodiments wherein the ‘click-on’ fluorogenic reporter is alkyne based (e.g., 7-ethynyl coumarin, 2-ethynyl benzothiazole, and 6-ethynyl napthalimide), the functionalized small molecule is an azido functionalized small molecule (e.g., wherein the alkyne interacts with the azido to develop a triazole ring thereby permitting the ‘click-on’ fluorogenic reporter to fluoresce). Thus, fluorescence from the fluorogenic dendrimer reporters is quenched until it binds with the respective functionalized small molecule (e.g., generating a triazole ring and resulting in detectable fluorescence) thereby permitting the tracking and monitoring of the small molecule and any biological process associated with such small molecule.

Experiments conducted during the course of developing embodiments for the present invention describe the synthesis and testing of a fluorogenic ‘click’ dendrimer reporter to monitor cellular processes. The reporter system consists of a polyamidoamine (PAMAM) dendrimer conjugated with 3-azido-7-hydroxy coumarin. After the copper (I)-catalyzed azide-alkyne cycloaddition reaction (‘click’ reaction) with alkyne-derivatized target molecules, the natively non-fluorescent construct was shown to have a strong enhancement in fluorescence. This fluorogenic dendrimer reporters were shown to be used to efficiently monitor biological processes and the specificity afforded by the ‘click’ reaction greatly reduced background noise and enhanced assay flexibility. Moreover, such experiments demonstrated that the fluorogenic dendrimer reporters (e.g., dendrimers conjugated with 3-azido coumarin molecules) have (in comparison to non-dendrimer based reporters utilizing coumarin) (1) improved aqueous solubility, (2) the ability to tune signal-to-noise with reduced background fluorescence in biological matrices, and (3) enhanced assay throughput owing to the reductions in purifications steps.

Accordingly, in certain embodiments, the present invention provides compositions comprising a dendrimer nanoparticle conjugated with a fluorogenic reporter. In some embodiments, the dendrimer nanoparticle is a PAMAM dendrimer.

In some embodiments, the fluorogenic reporter is conjugated with an azido chemical moiety. In some embodiments, the fluorogenic reporter conjugated with an azido chemical moiety is selected from the group consisting of 3-azido coumarin, 9-azido anthracene, and 6-azido napthalimide. In some embodiments, the fluorogenic reporter conjugated with an azido chemical moiety exhibits diminished fluorescence while conjugated with the dendrimer nanoparticle. In sonic embodiments, the detectable fluorescence from the formation of the triazole ring is approximately 20 fold higher in comparison to the diminished fluorescence.

In some embodiments, wherein upon interaction with an alkyne functionalized small molecule (e.g., the functionalized small molecule having an alkyne group or a cycloalkyne group) via a 1,3-dipolar cycloaddition reaction a triazole ring is formed, wherein the formation of the triazole ring results in detectable fluorescence from the ‘click-on’ fluorogenic reporter.

In sonic embodiments, the fluorogenic reporter is conjugated with an alkyne chemical moiety. In some embodiments, the fluorogenic reporter conjugated with an alkyne chemical moiety is selected from the group consisting of 7-ethynyl coumarin, 2-ethynyl benzothiazole, and 6-ethynyl napthalimide. In some embodiments, the fluorogenic reporter conjugated with an azido chemical moiety exhibits diminished fluorescence while conjugated with the dendrimer nanoparticle. In some embodiments, the detectable fluorescence from the formation of the triazole ring is approximately 20 fold higher in comparison to the diminished fluorescence. In some embodiments, wherein upon interaction with an azido functionalized small molecule via a 1,3-dipolar cycloaddition reaction a triazole ring is formed, wherein the formation of the triazole ring results in detectable fluorescence from the ‘click-on’ fluorogenic reporter.

In some embodiments, the dendrimer nanoparticle is further conjugated with one or more additional functional agents. In some embodiments, the additional functional agents are independently selected from the group consisting of a therapeutic agent, a targeting agent, an imaging agent, and a trigger agent.

In certain embodiments, the present invention provides methods of tracking a small molecule within a biological sample, comprising providing a dendrimer nanoparticle and a functionalized small molecule, wherein either i) the dendrimer nanoparticle is conjugated with an azido based ‘click-on’ fluorogenic reporter and the functionalized small molecule is an alkyne functionalized small molecule, or ii) the dendrimer nanoparticle is conjugated with an alkyne based ‘click-on’ fluorogenic reporter and the functionalized small molecule is an azido functionalized small molecule, and introducing the dendrimer nanoparticle and functionalized small molecule into a biological sample, detecting fluorescence from the dendrimer nanoparticle upon interaction between the dendrimer nanoparticle with the functionalized small molecule, wherein the interaction results in formation of a triazole ring via a 1,3-dipolar cycloaddition reaction, wherein the formation of the triazole ring results in detectable fluorescence from the ‘click-on’ fluorogenic reporter, and tracking the functionalized small molecule via fluorescence from the ‘click-on’ fluorescence reporter.

In some embodiments, the dendrimer nanoparticle is a PAMAM dendrimer.

In some embodiments, the azido based ‘click-on’ fluorogenic reporter is selected from the group consisting of 3-azido coumarin, 9-azido anthracene, and 6-azido napthalimide. In some embodiments, the azido based ‘click-on’ fluorogenic reporter exhibits diminished fluorescence while conjugated with the dendrimer nanoparticle. In some embodiments, the detectable fluorescence from the formation of the triazole ring is approximately 20 fold higher in comparison to the diminished fluorescence.

In some embodiments, the alkyne based ‘click-on’ fluorogenic reporter is selected from the group consisting of 7-ethynyl coumarin, 2-ethynyl benzothiazole, and 6-ethynyl napthalimide. In some embodiments, the alkyne based ‘click-on’ fluorogenic reporter exhibits diminished fluorescence while conjugated with the dendrimer nanoparticle. In some embodiments, the detectable fluorescence from the formation of the triazole ring is approximately 20 fold higher in comparison to the diminished fluorescence.

In sonic embodiments, the dendrimer nanoparticle is further conjugated with one or more additional functional agents. In some embodiments, the additional functional agents are independently selected from the group consisting of a therapeutic agent, a targeting agent, an imaging agent, and a trigger agent.

In some embodiments, the functionalized small molecule is selected from the group consisting of a protein, a polysaccharide, a carbohydrate, a lipid, a nucleic acid, an oligonucleotide, and a metabolite.

In some embodiments, the biological sample is within an in vivo setting, an ex vivo setting, or an in vitro setting.

In certain embodiments, the present invention provides methods of monitoring a biological process within a biological sample, comprising providing a dendrimer nanoparticle and a functionalized small molecule, wherein either i) the dendrimer nanoparticle is conjugated with an azido based ‘click-on’ fluorogenic reporter and the functionalized small molecule is an alkyne functionalized small molecule, or ii) the dendrimer nanoparticle is conjugated with an alkyne: based ‘click-on’ fluorogenic reporter and the functionalized small molecule is an azido functionalized small molecule, and introducing the dendrimer nanoparticle and functionalized small molecule into a biological sample, detecting fluorescence from the dendrimer nanoparticle upon interaction between the dendrimer nanoparticle with the functionalized small molecule, wherein the interaction results in formation of a triazole ring via a 1,3-dipolar cycloaddition reaction, wherein the formation of the triazole ring results in detectable fluorescence from the ‘click-on’ fluorogenic reporter, tracking the functionalized small molecule via fluorescence from the ‘click-on’ fluorescence reporter, and monitoring a biological process associated with the functionalized small molecule through tracking the delectable fluorescence.

In some embodiments, the dendrimer nanoparticle is a PAMAM dendrimer.

In some embodiments, the azido based ‘click-on’ fluorogenic reporter is selected from the group consisting of 3-azido coumarin, 9-azido anthracene, and 6-azido napthalimide. In some embodiments, the azido based ‘click-on’ fluorogenic reporter exhibits diminished fluorescence while conjugated with the dendrimer nanoparticle. In some embodiments, the detectable fluorescence from the formation of the triazole ring is approximately 20 fold higher in comparison to the diminished fluorescence.

In some embodiments, the alkyne based ‘click-on’ fluorogenic reporter is selected from the group consisting of 7-ethynyl coumarin, 2-ethynyl benzothiazole, and 6-ethynyl napthalimide. In some embodiments, the alkyne based ‘click-on’ fluorogenic reporter exhibits diminished fluorescence while conjugated with the dendrimer nanoparticle. In some embodiments, the detectable fluorescence from the formation of the triazole ring is approximately 20 fold higher in comparison to the diminished fluorescence.

In some embodiments, the dendrimer nanoparticle is further conjugated with one or more additional functional agents. In some embodiments, the additional functional agents are independently selected from the group consisting of a therapeutic agent, a targeting agent, an imaging agent, and a trigger agent.

In some embodiments, the functionalized small molecule is selected from the group consisting of a protein, a polysaccharide, a carbohydrate, a lipid, a nucleic acid, an oligonucleotide, and a metabolite.

In some embodiments, the biological sample is within an in vivo setting, air ex vivo setting, or an in vitro setting.

In some embodiments, the biological process is selected from the group consisting of nucleic acid related processes, cellular proliferation processes, drug metabolism processes, gene expression processes, protein modification processes, protein interaction processes, physiological processes, reproductive processes, digestion related processes, fermentation related processes, fertilization related processes, germination related processes, tropism related processes, hybridization related processes, metamorphosis related processes, photosynthesis related processes, and transpiration related processes.

Additional embodiments will be apparent to persons skilled in the relevant art based on the teachings contained herein.

### DESCRIPTION OF THE DRAWINGS

FIG. 1 shows emission spectra of Coumarin reporters after ‘click’ reaction: (a) G5-Coumarin 3 and G5-Coumarin-Click (red) (λₑₓ=360 nm); (b) 3-azido-7-hydroxy-coumarin and 3-azido-7-hydroxy-coumarin-Click (black) (λₑₓ=340 nm). G5-Coumarin 3 and 3-azido-7-hydroxy-coumarin were compared at same coumarin molar concentrations.

FIG. 2 shows monitoring cellular proliferation using fluorogenic reporters through detection of EdU. KB cells were incubated in media with EdU for 5 hours. Incorporated EdU was detected using either the coumarin reporter or the dendrimer reporter and compared to the standard “click” reporter, AF-647. L=low concentration and H=high concentration; No=no washes and Standard=2 wash steps. Results are representative of 3 independent experiments. Data represented as means with SEM.

Figure shows 1H NMR of G5-Coumarin 3.

### Definitions

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

As used herein, the term “nanodevice” or “nanodevices” refer, generally, to compositions comprising dendrimers of the present invention. As such, a nanodevice may refer to a composition comprising a dendrimer of the present invention that may contain one or more ligands, linkers, and/or functional groups (e.g., a therapeutic agent, a targeting agent, a trigger agent, an imaging agent) conjugated to the dendrimer.

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076). Similarly, the conjugate may comprise a cleavable linkage present linkage between the dendrimer and functional group, or, may comprise a cleavable linkage present in the polymer itself (See, e.g., U.S. Pat. App. Nos. 20050158273 and 20050181449).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “Baker-Huang dendrimer” or “Baker-Huang PAMAM dendrimer” refers to a dendrimer comprised of branching units of structure:

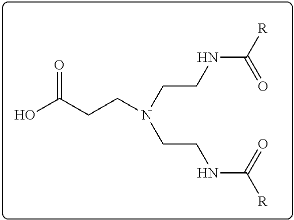

wherein R comprises a carbon-containing functional group (e.g., CF₃). In some embodiments, the branching unit is activated to its HNS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in Scheme 2). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines. In preferred embodiments, a Bake-Huang dendrimer is synthesized by convergent synthesis methods.

As used herein, the term “click chemistry” refers to chemistry tailored to generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362)).

As used herein, the term “triazine” refers to a compound comprising a ring structure bearing three nitrogen atoms. In some embodiments, the ring structure is six-membered (e.g., the molecular formula comprises C₃H₃N₃). In some embodiments, the ring is a conjugated system. Triazine moieties with six-membered rings may have nitrogen atoms at any possible placement so long as three nitrogen atoms occur in the ring (e.g., 1,2,3-triazine; 1,2,4-triazine, 1,3,5-triazine, 1,2,5-triazine, 1,2,6-triazine, etc.)

As used herein, the term “scaffold” refers to a compound to which other moieties are attached (e.g., conjugated). In some embodiments, a scaffold is conjugated to bioactive functional conjugates (e.g., a therapeutic agent, a targeting agent, a trigger agent, an imaging agent). In some embodiments, a scaffold is conjugated to a dendrimer (e.g., a PAMAM dendrimer). In some embodiments, conjugation of a scaffold to a dendrimer and/or a functional conjugate(s) is direct, while in other embodiments conjugation of a scaffold to a dendrimer and/or a functional conjugate(s) is indirect, e.g., an intervening linker is present between the scaffold compound and the dendrimer, and/or the scaffold and the functional conjugate(s).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, conjugation between a dendrimer (e.g., a terminal arm of a dendrimer) and a functional ligand is accomplished during a “one-pot” reaction. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino)pyridine) (see, e.g., International Patent Application No. PCT/U52010/042556).

As used herein, the term “solvent” refers to a medium in which a reaction is conducted. Solvents may be liquid but are not limited to liquid form. Solvent categories include but are not limited to nonpolar, polar, protic, and aprotic.

As used herein, the term “dialysis” refers to a purification method in which the solution surrounding a substance is exchanged over time with another solution. Dialysis is generally performed in liquid phase by placing a sample in a chamber, tubing, or other device with a selectively permeable membrane. In some embodiments, the selectively permeable membrane is cellulose membrane. In some embodiments, dialysis is performed for the purpose of buffer exchange. In some embodiments, dialysis may achieve concentration of the original sample volume. In some embodiments, dialysis may achieve dilution of the original sample volume.

As used herein, the term “precipitation” refers to purification of a substance by causing it to take solid form, usually within a liquid context. Precipitation may then allow collection of the purified substance by physical handling, e.g. centrifugation or filtration.

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino)pyridine, or dicyclohexylcarbodiimide and 4-(dimethylamino)pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “ligand” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound (e.g., triazine scaffold) intervenes. In preferred embodiments, ligands have functional utility for specific applications, e.g., for therapeutic, targeting, imaging, or drug delivery function(s). The terms “ligand”, “conjugate”, and “functional group” may be used interchangeably.

As used herein, the term “alkyne functionalized small molecule,” or similar term, refers to a small molecule associated with (e.g., conjugated with) an alkyne moiety. Examples such alkyne moieties include, but are not limited to, alkyne groups and cycloalkyne groups.

### DETAILED DESCRIPTION OF HE INVENTION

Monitoring biomolecules in living systems using bioorthogonal chemical reporters has drawn much attention (see, e.g., Prescher, J. A.; Bertozzi, C. R. Nat Chem Biol. 2005, 1, 13; Link, A. J.; Vink, M. K. S.; Tirrell, D. A. J Am Chem Soc 2004, 126, 10598; Griffin, B. A.; Adams, S. R.; Tsien, R. Y. Science 1998, 281, 269; Zhang, Z. W.; Smith, B. A. C.; Wang, L.; Brock, A.; Cho, C.; Schultz, P. G. Biochemistry-Us 2003, 42, 6735; Kho, Y.; Kim, S. C.; Jiang, C.; Barma, D.; Kwon, S. W.; Cheng, J. K.; Jaunbergs, J.; Weinbaum, C.; Tamanoi, F.; Falck, J.; Zhao, Y. M. P Natl Acad Sci USA 2004, 101, 12479; Mahal, L. K.; Yarema, K. J.; Bertozzi, C. R. Science 1997, 276, 1125). The introduction of such reporter tags rely on selective and fast reaction, under physiological conditions, and inertness to the surrounding biological functionalities. Copper (I)-catalyzed azide-alkyne cycloaddition reaction (‘click’ reaction) has provided investigators with a powerful, chemoselective tool to conjugate chemical reporters that overcomes many of the existing reporter limitations (see, e.g., Kolb, H. C.; Finn, M. G.; Sharpless, K. B. Angewandte Chemie-International Edition 2001, 40, 2004; Lewis, W. G.; Green, L. G.; Grynszpan, F.; Radic, Z.; Curlier, P. R.; Taylor, P.; Finn, M. G.; Sharpless, K. B. Angewandte Chemie-International Edition 2002, 41, 1053).

Click chemistry involves, for example, the coupling of two different moieties via a 1,3-dipolar cycloaddition reaction between an alkyne moiety (or equivalent thereof) on the surface of the first moeity and an azide moiety (or equivalent thereof) on the second moiety. Click chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932).

‘Click’ fluorescent reporters have been used to label a wide spectrum molecules including nucleotides, amino acids, lipids, and monosaccharides (see, e.g., Jewett, J. C.; Bertozzi, C. R. Chem Soc Rev 2010, 39, 1272). These ‘click’ fluorescent reporters utilize a unique chemical handle (azide or alkyne reactive group) that has been introduced onto the target molecule using the cells own biosynthetic machinery. The target molecule is detected by the highly selective ligation of a ‘click’ fluorescent reporter to the molecule's chemical handle. Because of the unobtrusive nature of the azide and alkyne handles, they can be easily incorporated into target molecules without perturbing the targets biological function. Additionally, because these reactive groups are only rarely present in biological systems, the ‘click’ reaction is highly specific and is well suited for monitoring of cellular processes such as DNA synthesis, protein translation, and post-translational modifications (see, e.g., Prescher, J. A.; Bertozzi, C. R. Nat Chem Biol 2005, 1, 13; Best, M. D. Biochemistry-Us 2009, 48, 6571; Kolb, H. C.; Sharpless, K. B. Drug Discov Today 2003, 8, 1128; Kele, P.; Li, X. H.; Link, M.; Nagy, K.; Herner, A.; Lorincz, K.; Beni, S.; Wolfbeis, O. S. Org Biomol Chem 2009, 7, 3486).

Although the use of bioorthogonal molecular profiling using ‘click’ fluorescent reporters has greatly expanded, the existing ‘click’ fluorescent reporters still have certain limitations including poor water solubility and high background noise (see, e.g., Jewett, J. C.; Bertozzi, C. R. Chem Soc Rev 2010, 39, 1272; Le Droumaguet, C.; Wang, C.; Wang, Q. Chem Soc Rev 2010, 39, 1233). Because typical fluorescent reporters fluoresce continuously and do not predictably change their emission properties after they have been conjugated, the unreacted reporter contributes to background noise and requires extensive purification steps limiting assay throughput. An alternative strategy that is not limited by the need for extensive purifications or background noise is to use fluorogenic chemical reporters, whose emission wavelength or intensity changes after conjugation to its target molecule (see, e.g., Goddard, J. P.; Reymond, J. L. Trends Biotechnol 2004, 22, 363). The unique properties of these fluorogenic reporters provide the opportunity to enhance signal-to-noise and to reduce the purification steps associated with existing fluorescent reporters. While this approach has obvious benefits, it has not been widely used because of the limited availability of fluorogenic reporters, their poor water solubility, and the lack of chemoselectivity in biological systems.

‘Click-on’ fluorogenic reporters take advantage of the electronic changes associated with the formation of the triazole ring during the ‘click’ reaction. Examples of ‘click-on’ fluorogenic reporters, include but are not limited to, azido based ‘click-on’ flurogenic reporters and alkyne based ‘click-on’ fluorogenic reporters (see, e.g., Qi, J., et al., 2011, Bioconjugate Chem. 22, 1758-1762). Examples of azido based ‘click-on’ fluorogenic reporers include, but are not limited to, 3-azido coumarin

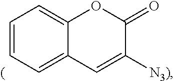

9-azido anthracene

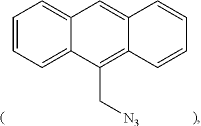 [SMILES: [N-]=[N+]=NCc1c2ccccc2cc2ccccc12]

and 6-azido napthalimide

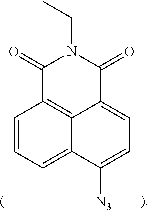 [SMILES: CCN1C(=O)c2cccc3c(N=[N+]=[N-])ccc(c23)C1=O]

Examples of alkyne based ‘click-on’ fluorogenic reporters includes, but are not limited to, 7-ethynyl coumarin

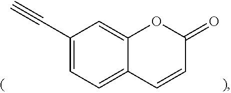

2-ethynyl benzothiazole

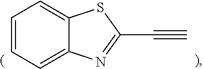

and 6-ethynyl napthalimide

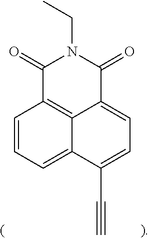 [SMILES: C#Cc1ccc2c3c(cccc13)C(=O)N(CC)C2=O]

The 3-azido coumarin small molecule, for example, has been developed and shown utility in labeling a range of molecules (see, e.g., Sivakumar, K.; Xie, F.; Cash, B. M.; Long, S.; Barnhill, H. N.; Wang, Q. Org Lett 2004, 6, 4603). The fluorescence properties of coumarin are strongly influenced by substituents at the 3 and 7-position. The introduction of an azido moiety at the 3-position quenches the coumarin fluorescence due to the electron donating properties of the azido group. After the ‘click’ reaction, the formation of the triazole ring delocalizes the electrons which restores the fluorescence of the coumarin (see, e.g., Le Droumaguet, C.; Wang, C.; Wang, Q. Chem Soc Rev 2010, 39, 1233; Qi, J. J.; Han, M. S.; Chang, Y. C.; Tung, C. H. Bioconjugate Chem 2011, 22, 1758).

These types of “click-on” fluorogenic reporters have significant potential in labeling applications but are still limited by their poor water solubility and incomplete fluorescence quenching in biological matrices.

To address these limitations while preserving the functionality of the ‘click-on’ flourogenic reporters, the present invention provides dendrimer nanoparticles conjugated with a ‘click-on’ fluorogenic reporter (e.g., 3-azido coumarin, 9-azido anthracene, 6-azido napthalimide, 7-ethynyl coumarin, 2-ethynyl benzothiazole, and 6-ethynyl napthalimide) thereby generating fluorogenic dendrimer reporters. Such fluorogenic dendrimer reporters are not limited to particular uses. In some embodiments, such fluorogenic dendrimer reporters (e.g., dendrimers conjugated with a ‘click-on’ fluorogenic reporter (e.g., 3-azido coumarin, 9-azido anthracene, 6-azido napthalimide, 7-ethynyl coumarin, 2-ethynyl benzothiazole, and 6-ethynyl napthalimide)) are used to track functionalized small molecules configured to interact with the ‘click-on’ fluorogenic reporter to develop a triazole ring, thereby permitting the ‘click-on’ fluorogenic reporter to fluoresce. As such, in embodiments wherein the ‘click-on’ fluorogenic reporter is azido based (e.g., 3-azido coumarin, 9-azido anthracene, 6-azido napthalimide), the functionalized small molecule is an alkyne functionalized small molecule (e.g., wherein the azido interacts with the alkyne to develop a triazole ring thereby permitting the ‘click-on’ fluorogenic reporter to fluoresce). In some embodiments, the alkyne within the alkyne functionalized small molecule is an alkyne group. In some embodiments, the alkyne within the alkyne: functionalized small molecule is a cycloalkyne group. Similarly, in embodiments wherein the ‘click-on’ fluorogenic reporter is alkyne based (e.g., 7-ethynyl coumarin, 2-ethynyl benzothiazole, and 6-ethynyl napthalimide), the functionalized small molecule is an azido functionalized small molecule (e.g., wherein the alkyne interacts with the azido to develop a triazole ring thereby permitting the ‘click-on’ fluorogenic reporter to fluoresce). Thus, fluorescence from the fluorogenic dendrimer reporters is quenched until it binds with the respective functionalized small molecule (e.g., generating a triazole ring and resulting in detectable fluorescence) thereby permitting the tracking and monitoring of the small molecule and any biological process associated with such small molecule.

In some embodiments, such fluorogenic dendrimer reporters are used to track and monitor biological processes (e.g., DNA synthesis, cellular proliferation, etc.) associated with such functionalized small molecules (e.g., alkyne functionalized small molecules or azido functionalized small molecules). Indeed, experiments conducted during the course of developing embodiments for the present invention demonstrated that dendrimers conjugated with 3-azido coumarin molecules were able to successfully monitor incorporation of for example, 5-ethynyl-2′-deoxyuridine (EdU) into newly synthesized DNA, as a surrogate marker of cellular proliferation. Moreover, such experiments demonstrated that the fluorogenic dendrimer reporters (e.g., dendrimers conjugated with 3-azido coumarin molecules) have (in comparison to non-dendrimer based reporters utilizing coumarin) (1) improved aqueous solubility, (2) the ability to tune signal-to-noise with reduced background fluorescence in biological matrices, and (3) enhanced assay throughput owing to the reductions in purifications steps.

The fluorogenic dendrimer reporters (e.g., dendrimers conjugated with ‘click-on’ fluorogenic reporters) are not limited to tracking particular functionalized small molecules (e.g., alkyne-functionalized small molecules or azido-functionalized small molecules). Examples of applicable functionalized small molecules include, but are not limited to, any organic molecule that is produced by a living organism or any artificially produced derivatives of such compounds, including large polymeric molecules such as proteins, polysaccharides, carbohydrates, lipids, nucleic acids and oligonucleotides as well as primary metabolites, secondary metabolites, and natural products. In some embodiments, the alkyne-functionalized small molecule is associated with a cellular process of interest (e.g., DNA synthesis, cellular proliferation, etc.). In some embodiments, the small molecule is a nucleotide, amino acid, lipid, and/or a monosaccharide.

In some embodiments, the fluorogenic dendrimer reporters (e.g., dendrimers conjugated with a ‘click-on’ fluorogenic reporter) are used to monitor a biological process via tracking of a functionalized small molecule (e.g., alkyne-functionalized small molecule or azido-functionalized small molecule). Examples of such biological processes include, but are not limited to, any processes associated with a particular functionalized small molecule (e.g., alkyne-functionalized small molecules or azido-functionalized small molecules). Examples of such biological processes include, but are not limited to, nucleic acid related processes (e.g., DNA synthesis), cellular proliferation processes, drug metabolism, gene expression, protein modification or interaction with a protein or substrate molecule, physiological processes (e.g., processes specifically pertinent to the functioning of integrated living units: cells, tissues, organs, and organisms), reproductive processes, digestion related processes, fermentation related processes, fertilization related processes, germination related processes, tropism related processes, hybridization related processes, metamorphosis related processes, photosynthesis related processes, and/or transpiration related processes. In some embodiments, the fluorogenic dendrimer are used to monitor a biological process via tracking of a particular functionalized small molecule within any type of setting (e.g., in vitro setting, in vivo setting, ex vivo setting).

The fluorogenic dendrimer reporters (e.g., dendrimers conjugated with a ‘click-on’ fluorogenic reporter) are not limited to utilizing a particular type of dendrimer nanoparticle. Dendrimeric polymers have been described extensively (see, e.g., Tomalia, Advanced Materials 6:529 (1994); Angew, Chem. Int. Ed. Engl., 29:138 (1990)). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179). In preferred embodiments, the protected core diamine is NH₂—CH₂—CH₂-NHPG. Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process. In some embodiments, the PAMAM dendrimers are “Baker-Huang dendrimers” or “Baker-Huang PAMAM dendrimers” (see, e.g., U.S. Provisional Patent Application Ser. No. 61/251,244).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. Patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense Mar polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per, unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH). They are water soluble, biocompatible, and cleared from the blood through the kidneys (Peer et al. (2007) Nat. Nanotechnol. 2:751-760) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462), and imaging applications (Kobayashi et al. (2003) Bioconj. Chem. 14:388-394).

The use of dendrimers as metal carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polycation non-covalently coupled to the poly-nucleotide.

In some embodiments, the fluorogenic dendrimer reporters (e.g., dendrimers conjugated with a ‘click-on’ fluorogenic reporter comprise PAMAM dendrimers.

The fluorogenic dendrimer reporters of the present invention are not limited to conjugation with a particular fluorogenic reporting agents. As noted, ‘click-on’ fluorogenic reporters take advantage of the electronic changes associated with the formation of the triazole ring during the ‘click’ reaction. Examples of ‘click-on’ fluorogenic reporters for conjugation with a dendrimer to generate a fluorogenic dendrimer reporter, include but are not limited to, azido based ‘click-on’ flurogenic reporters and alkyne based ‘click-on’ fluorogenic reporters (see, e.g., Qi, J., et al., 2011, Bioconjugate Chem. 22, 1758-1762). Examples of azido based ‘click-on’ fluorogenic reporers include, but are not limited to, 3-azido coumarin, 9-azido anthracene, 6-azido napthalimide, 7-ethynyl coumarin, 2-ethynyl benzothiazole, and 6-ethynyl napthalimide.

The fluorogenic dendrimer reporters of the present invention are not limited to a particular manner of conjugation with a ‘click-on’ fluorogenic reporter. In some embodiments, the fluorogenic dendrimer reporters are conjugated with the ‘click-on’ fluorogenic reporters (e.g., 3-azido coumarin, 9-azido anthracene, 6-azido napthalimide, 7-ethynyl coumarin, 2-ethynyl benzothiazole, and 6-ethynyl napthalimide) via linkage agents. Examples of such linkage agents include, but are not limited to, thiol groups, diene groups, dieneophile groups, and alkene groups.

In some embodiments, conjugation between the fluorogenic dendrimer reporters (e.g., dendrimers conjugated with 3-azido coumarin molecules) and the ‘click-on’ fluorogenic reporters are accomplished via classical multi-step conjugation. Classical multi-step conjugation strategies used during the synthesis of functionalized dendrimers generate a stochastic distribution of products with differing numbers of ligands attached per dendrimer molecule, thereby creating a population of dendrimers with a wide distribution in the numbers of ligands attached. The low structural uniformity of such dendrimer populations negatively affects properties such as therapeutic potency, pharmacokinetics, or effectiveness for multivalent targeting. Difficulties in quantifying and resolving such populations to yield samples with sufficient structural uniformity can pose challenges. However, in some embodiments, use of separation methods (e.g., reverse phase chromatography) customized for optimal separation of dendrimer populations in conjunction with peak fitting analysis methods allows isolation and identification of subpopulations of functionalized dendrimers with high structural uniformity (see, e.g., U.S. Provisional Pat. App. No. 61/237,172)).

In some embodiments, conjugation between the fluorogenic dendrimer reporters (e.g., dendrimers conjugated with 3-azido coumarin molecules) and the ‘click-on’ fluorogenic reporters are accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a ‘click-on’ fluorogenic reporter) (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino)pyridine) (see, e.g., international Patent Application No. PCT/US2010/042556).

The fluorogenic dendrimer reporters are not limited to conjugation with a particular number of ‘click-on’ fluorogenic reporters. For example, in some embodiments, the fluorogenic dendrimer reporters have between 1 and 128 ‘click-on’ fluorogenic reporters. In some embodiments, the number of fluorogenic reporting agents (e.g., 3-azido coumarin, 9-azido anthracene, 6-azido napthalimide, 7-ethynyl coumarin, 2-ethynyl benzothiazole, and 6-ethynyl napthalimide) conjugated to the dendrimer nanoparticle is sufficient to accomplish tracking of functionalized small molecules (e.g., alkyne functionalized small molecules or azido functionalized small molecules) and cellular processes associated with such functionalized small molecules (e.g., DNA synthesis, cellular proliferation, etc.).

The fluorogenic dendrimer reporters of the present invention may be characterized for size and structural uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC), size exclusion chromatography (SEC) (equipped with multi-angle laser light scattering, dual UV and refractive index detectors), gel permeation chromatography (GPC), capillary electrophoresis and get electrophoresis. These analytical methods assure the uniformity of the dendrimer population and are important in the quality control of dendrimer production for eventual use, for example, in in vivo applications.

In some embodiments, the fluorogenic dendrimer reporters are further conjugated with additional functional agents (e.g., additional imaging agents, targeting agents, therapeutic agents). In some embodiments, such additional functional agents are conjugated with the dendrimer nanoparticle via linkage agents. Examples of such linkage agents include, but are not limited to, thiol groups, diene groups, dieneophile groups, and alkene groups.

Examples of therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agents are effective in treating cancer (see, e.g., U.S. Pat. Nos. 6,471,968, 7,078,461, and U.S. patent application Ser. Nos. 09/940,143, 10/431,682, 11/503,742, 11/661,465, 11/523,509, 12/403,179, 12/106,876, and 11/827,637; and U.S. Provisional Patent Application Ser. Nos. 61/256,759, 61/140,840, 61/091,608, 61/097,780, 61/101,461, 61/237,172, 61/229,168, 61/221,596, and 61/251,244).

In some embodiments, the therapeutic agents may be any agent selected from the group comprising, but not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein.

In some embodiments, the fluorogenic dendrimer reporters are conjugated with a targeting agent. The present invention is not limited to any particular targeting agent. In some embodiments, targeting agents are conjugated to the dendrimer (e.g., directly or indirectly) for delivery to desired body regions (e.g., to the central nervous system (CNS); to a tumor). The targeting agents are not limited to targeting specific body regions.

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, multifunctional dendrimers can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some embodiments, the targeting agent is an antibody. In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443); human carcinoma antigen (See, e.g., U.S. Pat. Nos. 5,693,763; 5,545,530; and 5,808,005); TP1 and TP3 antigens from osteocarcinoma cells (See, e.g., U.S. Pat. No. 5,855,866)); Thomsen-Friedenreich (TF) antigen from adenocarcinoma cells (See, e.g., U.S. Pat. No. 5,110,911)); “KC-4 antigen” from human prostrate adenocarcinoma (See, e.g., U.S. Pat. Nos. 4,708,930 and 4,743,543); a human colorectal cancer antigen (See, e.g., U.S. Pat. No. 4,921,789)); CA125 antigen from cystadenocarcinoma (See, e.g., U.S. Pat. No. 4,921,790)); DF3 antigen from human breast carcinoma (See, e.g., U.S. Pat. Nos. 4,963,484 and 5,053,489); a human breast tumor antigen (See, e.g., U.S. Pat. No. 4,939,240); p97 antigen of human melanoma (See, e.g., U.S. Pat. No. 4,918,164); carcinoma or orosomucoid-related antigen (CORA) (See, e.g., U.S. Pat. No. 4,914,021)); a human pulmonary carcinoma antigen that reacts with human squamous cell lung carcinoma but not with human small cell lung carcinoma (See, e.g., U.S. Pat. No. 4,892,935)); T and Tn haptens in glycoproteins of human breast carcinoma (See, e.g., Springer et al., Carbohydr. Res. 178:271-292 (1988))), MSA breast carcinoma glycoprotein termed (See, e.g., Tjandra et al., Br. J. Surg. 75:811-817 (1988))); MFGM breast carcinoma antigen (See, e.g., Ishida et al., Tumor Biol., 10:12-22 (1989))); DU-PAN-2 pancreatic carcinoma antigen (See, e.g., Lan et al., Cancer Res, 45:305-310 (1985))); CA125 ovarian carcinoma antigen (See, e.g., Hanisch et al., Carbohydr. Res. 178:29-47 (1988))); YH206 lung carcinoma antigen (See, e.g., Hinoda et al., (1988) Cancer J. 42:653-658 (1988))).

In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006, 121(2): p. 144-158). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214)). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR) (SEQ ID NO: 1)) (see, e.g., Liu, J. K., et al., Neurobiology of Disease, 2005. 19(3): p. 407-418)).

In some embodiments, the fluorogenic dendrimer reporters are conjugated with additional imaging agents. Examples of such additional imaging agents include, but are not limited to, molecular dyes, fluorescein isothiocyanate (FITC), 6-TAMARA, acridine orange, and cis-parinaric acid. In some embodiments, the imaging agents are moleculear dyes from the alexa fluor (Molecular Probes) family of molecular dyes. For example, examples of imaging agents include, but are not limited to, Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™5, PerCP, PerCP-Cy™5.5, PE-Cy™7, APC-Cy 7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight® 550, DyLight 594, DyLight 633, DyLight® 650, DyLight 680, DyLight 755, DyLight 800, Tracy 645, Tracy 652, Atto 488, Atto 520, Atto 532, Alto Rho6G, Alto 550, Alto 565, Alto 590, Alto 594, Alto 633, Alto Rho11, Atto Rho14, Atto 647, Atto 647N, Atto 655, Atto 680, Atto 700, CF™350, CF™405S, CF™405M, CF™488A, CF™543, CF™555, CF™568, CF™594, CF™620R, CF™633, CF™640R, CF™647, CF™660, CF™660R, CF™680, CF™680R, CF™750, CF™770, and CF™790. In some embodiments, the additional, imaging agent is a mass-spec label selected from the group consisting of 139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb.

In some embodiments, chelated paramagnetic ions, such as Gd(III)-diethylenetriaminepentaacetic acid (Gd(III)-DTPA), are conjugated to the fluorogenic dendrimer reporters (e.g., dendrimers conjugated with 3-azido coumarin molecules). Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof.

Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

In some embodiments, the fluorogenic dendrimer reporters allow functional microscopic imaging of tumors and provide improved methods for imaging. The methods find use in vivo, in vitro, and ex vivo. For example, in one embodiment, dendrimer functional groups are designed to emit light or other detectable signals upon exposure to light. Although the labeled functional groups may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997))). In embodiments where the imaging agents are present in deeper tissue, longer wavelengths in the Near-infrared (NMR) are used (See e.g., Lester et al., Cell Mol. Biol. 44:29 (1998))). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce when excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In some embodiments, the additional functional agent is conjugated with the dendrimer via a trigger agent. The present invention is not limited to particular types or kinds of trigger agents.

In some embodiments, sustained release (e.g., slow release over a period of 24-48 hours) of the ligand (e.g., therapeutic agent) is accomplished through conjugating the therapeutic agent (e.g., directly) (e.g., indirectly through one or more additional functional groups) to a trigger agent that slowly degrades in a biological system (e.g., amide linkage, ester linkage, ether linkage). In some embodiments, constitutively active release of a therapeutic agent is accomplished through conjugating the therapeutic agent to a trigger agent that renders the therapeutic agent constitutively active in a biological system (e.g., amide linkage, ether linkage).

In some embodiments, release of a therapeutic agent under specific conditions is accomplished through conjugating the therapeutic agent (e.g., directly) (e.g., indirectly through one or more additional functional groups) to a trigger agent that degrades under such specific conditions (e.g., through activation of a trigger molecule under specific conditions that leads to release of the therapeutic agent). For example, once a conjugate (e.g., a therapeutic agent conjugated with a trigger agent and a targeting agent) arrives at a target site in a subject (e.g., a tumor, or a site of inflammation), components in the target site (e.g., a tumor associated factor, or an inflammatory or pain associated factor) interact with the trigger agent thereby initiating cleavage of the therapeutic agent from the trigger agent. In some embodiments, the trigger agent is configured to degrade (e.g., release the therapeutic agent) upon exposure to a tumor-associated factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides a therapeutic age conjugated with a trigger agent that is sensitive to (e.g., is cleaved by) hypoxia indolequinone). Hypoxia is a feature of several disease states, including cancer, inflammation and rheumatoid arthritis, as well as an indicator of respiratory depression (e.g., resulting from analgesic drugs).

Advances in the chemistry of bioreductive drug activation have led to the design of various hypoxia-selective drug delivery systems in which the pharmacophores of drugs are masked by reductively cleaved groups. In some embodiments, the trigger agent is utilizes a quinone, N-oxide and/or (hetero)aromatic nitro groups. For example, a quinone present in a conjugate is reduced to phenol under hypoxia conditions, with spontaneous formation of lactone that serves as a driving force for drug release. In some embodiments, a heteroaromatic nitro compound present in a conjugate (e.g., a therapeutic agent conjugated (e.g., directly or indirectly) with a trigger agent) is reduced to either an amine or a hydroxylamine, thereby triggering the spontaneous release of a therapeutic agent. In some embodiments, the trigger agent degrades upon detection of reduced pO2 concentrations (e.g., through use of a redox linker).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M. D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20,): p. 4071-4080; Zhang, Z., et al., Organic & Biomolecular Chemistry, 2005. 3(10): p. 1905-1910). Several such hypoxia activated pro-drugs have been advanced to clinical investigations, and work in relevant oxygen concentrations to prevent cerebral damage. The present invention is not limited to particular hypoxia activated trigger agents. In some embodiments, the hypoxia activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770).

In sonic embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see, e.g., Damen. E. W. P., et al., Bioorganic & Medicinal Chemistry, 2002. 10(1): p. 71-77)). For example, in such embodiments, the antagonist is only active when released during hypoxia to prevent respiratory failure.

In sonic embodiments, the trigger agent is sensitive to (e.g., is cleaved by) and/or associates with a protease. The present invention is not limited to any particular protease. In some embodiments, the protease is a cathepsin, in some embodiments, a trigger comprises a Lys-Phe-PABC moiety (e.g., that acts as a trigger). In sonic embodiments, a Lys-Phe-PABC moiety linked to doxorubicin, mitomycin C, and paclitaxel are utilized as a trigger-therapeutic conjugate in a dendrimer conjugate provided herein (e.g., that serve as substrates for lysosomal cathepsin B or other proteases expressed (e.g., overexpressed) in tumor cells. In some embodiments, utilization of a 1,6-elimination spacer/linker is utilized (e.g., to permit release of therapeutic drug post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloprotease (MMP). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., a tumor cell)).

In some embodiments, the trigger agent is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a dendrimer conjugate). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, conjugation between the fluorogenic dendrimer reporters and additional functional agents are accomplished during a “one-pot” reaction (see, e.g., International Patent Application No. PCT/US2010/042556). In some embodiments, conjugation between the fluorogenic dendrimer reporters and additional functional agents are accomplished via classical multi-step conjugation (see, e.g., U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, 61/736,225, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023).

In certain embodiments, such methods and systems provide a dendrimer product made by the process comprising: a) conjugation of at least one ligand type to a dendrimer (e.g., ‘click-on’ fluorogenic reporter) to yield a population of ligand-conjugated dendrimers; b) separation of the population of ligand-conjugated dendrimers with reverse phase HPLC to result in subpopulations of ligand-conjugated dendrimers indicated by a chromatographic trace; and c) application of peak fitting analysis to the chromatographic trace to identify subpopulations of ligand-conjugated dendrimers wherein the structural uniformity of ligand conjugates per molecule of dendrimer within said subpopulation is, e.g., approximately 70% or more.

In some embodiments, the fluorogenic dendrimer reporters are used for the treatment of inflammatory diseases (e.g., fluorogenic dendrimer reporters conjugated with therapeutic agents configured for treating inflammatory diseases). Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyclitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodus, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

In some embodiments, the fluorogenic dendrimer reporters of the present invention configured for treating autoimmune disorders and/or inflammatory disorders (e.g., rheumatoid arthritis) are co-administered to a subject (e.g., a human suffering from an autoimmune disorder and/or an inflammatory disorder) a therapeutic agent configured for treating autoimmune disorders and/or inflammatory disorders (e.g., rheumatoid arthritis). Examples of such agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the medical condition and/or disease is pain (e.g., chronic pain, mild pain, recurring pain, severe pain, etc.). In some embodiments, the conjugated dendrimers of the present invention are configured to deliver pain relief agents to a subject. In some embodiments, the dendrimer conjugates are configured to deliver pain relief agents and pain relief agent antagonists to counter the side effects of pain relief agents. The dendrimer conjugates are not limited to treating a particular type of pain and/or pain resulting from a disease. Examples include, but are not limited to, pain resulting from trauma (e.g., trauma experienced on a battlefield, trauma experienced in an accident (e.g., car accident)). In some embodiments, the dendrimer conjugates of the present invention are configured such that they are readily cleared from the subject (e.g., so that there is little to no detectable toxicity at efficacious doses).

In some embodiments, the disease is cancer. The present invention is not limited by the type of cancer treated using the compositions and methods of the present invention. Indeed, a variety of cancer can be treated including, but not limited to, prostate cancer, colon cancer, breast cancer, lung cancer and epithelial cancer.

In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma., bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In some embodiments, the composition is co-administered with an anti-cancer agent. In some embodiments, the composition is co-administered with a pain relief agent. In some embodiments, the pain relief agents include, but are not limited to, analgesic drugs, anxiolytic drugs, anesthetic drugs, antipsychotic drugs, hypnotic drugs, sedative drugs, and muscle relaxant drugs. In some embodiments, the composition is co-administered with a pain relief agent antagonist.

Where clinical applications are contemplated, in some embodiments of the present invention, the fluorogenic dendrimer reporters are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the fluorogenic dendrimer reporters are used in conjunction with appropriate salts and buffers to render delivery of the compositions in a stable manner to allow for uptake by target cells. Buffers also are employed when the dendrimer conjugates are introduced into a patient. Aqueous compositions comprise an effective amount of the dendrimer conjugates to cells dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with vectors, cells, or tissues, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active fluorogenic dendrimer reporters may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating be active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, fluorogenic dendrimer reporters are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications arc available in a variety of physical forms, e.g., creams, gels or liquids, which depart from the classical concept of suppositories. In addition, suppositories may be used in connection with colon cancer. The dendrimer conjugates also may be formulated as inhalants for the treatment of lung cancer and such like.

In some embodiments, the present invention also provides kits comprising one or more of the reagents and tools necessary to generate a fluorogenic dendrimer reporters (e.g., dendrimers conjugated with 3-azido coumarin molecules) and alkyne-functionalized small molecules, and methods of using such dendrimers.

### EXAMPLES

The following examples arc provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, 61/736,225, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023.

### Example 2

This example describes fluorogenic dendrimer reporters of the present invention. The fluorogenic dendrimer reporters of the present invention are composed of a partially acetylated G5 PAMAM dendrimer (see, e.g., Majoros, I. J.; Thomas, T. P.; Mehta, C. B.; Baker, J. R. J Med Chem 2005, 48, 5892), a fluorogenic 3-azido-7-hydroxy coumarin dye, and a triazine ring linker (Scheme 1). The synthetic strategy is comprised of three parts: (1) conjugation of 3-azido-7-hydroxy coumarin to 2,4,6-trichloro-1,3,5-triazine at 0° C., triazine-coumarin 1; (2) G5 dendrimer was partially acetylated (70%) with acetic anhydride, G5-NHAc-NH₂ 2; (3) conjugation of triazine-coumarin to the partially acetylated G5 dendrimer at room temperature, G5-Coumarin 3. The ease of displacement of the chlorine atoms in 2,4,6-trichloro-1,3,5-triazine by various nucleophiles in a controlled manner makes this reagent useful to serve as a cross-linker between G5 dendrimer and 3-azido-7-hydroxy coumarin. After reacting with 2,4,6-trichloro-1,3,5-triazine, the Triazine-Coumarin dye 1 can be ligated to any macromolecule possessing a free amino group by simply mixing them together at room temperature. The final conjugate was characterized by analytical HPLC, NMR, and MALDI.

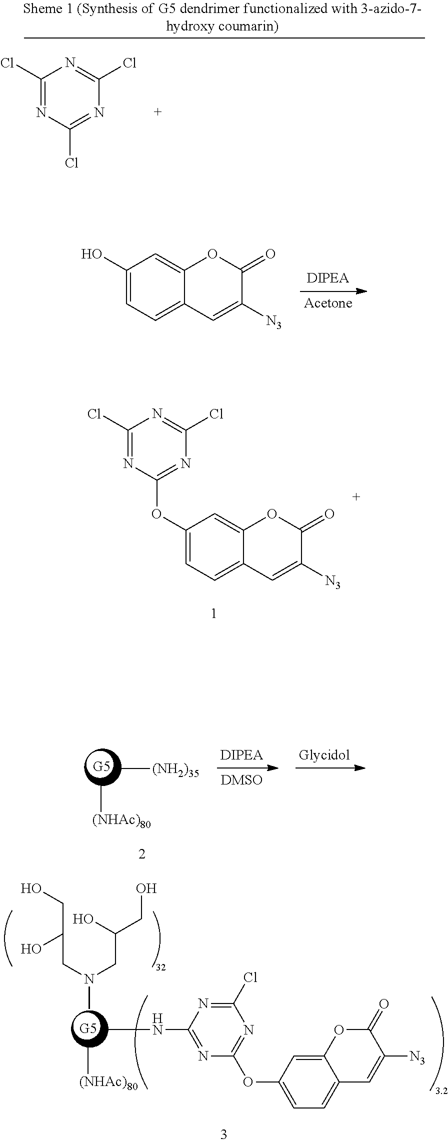

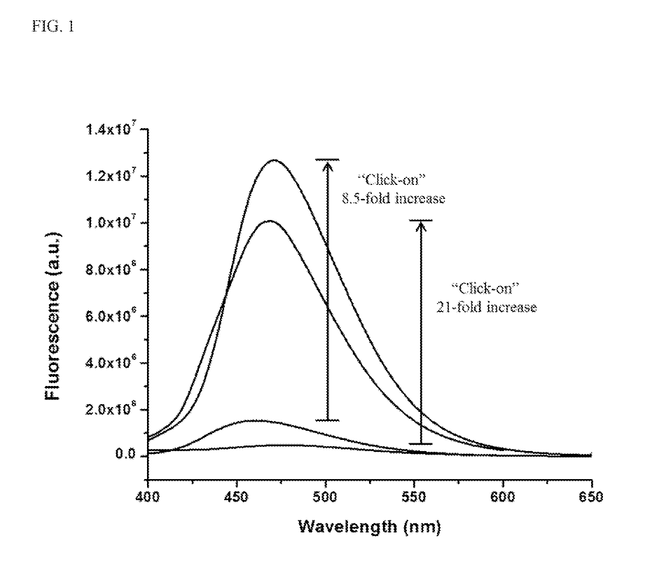

The fluorogenic properties of the small molecule and dendrimer coumarin reporters were first evaluated by performing the ‘click’ reaction with a representative alkyne-derivatized molecule, 3-(4-(prop-2-yn-1-yloxy)phenyl)propionic acid at room temperature for 12 hours (Scheme 2). Azidocoumarin itself shows very weak fluorescence and the ‘click’ product shows strong fluorescence. Indeed, the strong fluorescence was observed and both small molecule and dendrimer ‘click-on’ products showed similar fluorescence properties (λmax Em 470 nm). However, after attaching to the dendrimer, the absorbance of azidocoumarin has red-shifted 20 nm (λmax Ex 340 vs. 360 nm). Following ligation with the alkyne-derivatized molecule, the G5-Coumarin 3 increased its fluorescence 21-fold (FIG. 1). In contrast, the ‘click’ product from the small molecule 3-azido-7-hydroxy coumarin reaction with the alkyne-derivatized molecule demonstrated only an 8.5-fold increase in fluorescence intensity. This difference in fluorescent enhancement was primarily attributable to the decrease in the background fluorescence intensity of azidocoumarin on the dendrimer reporter. The structure-property relationships are still under investigation.

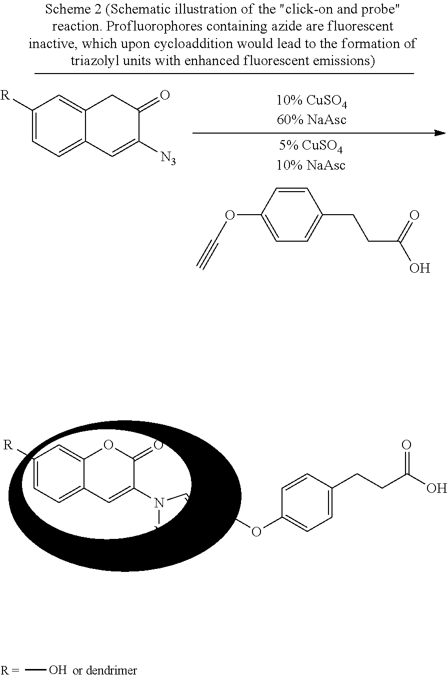

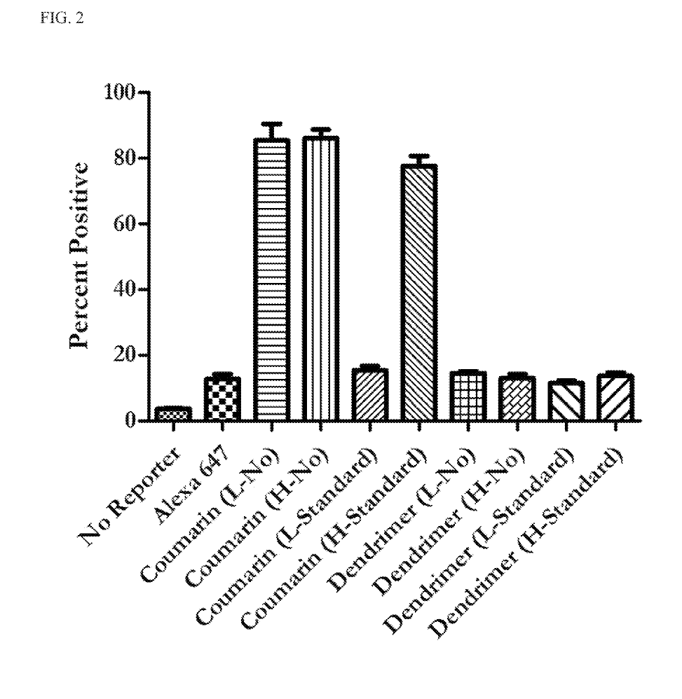

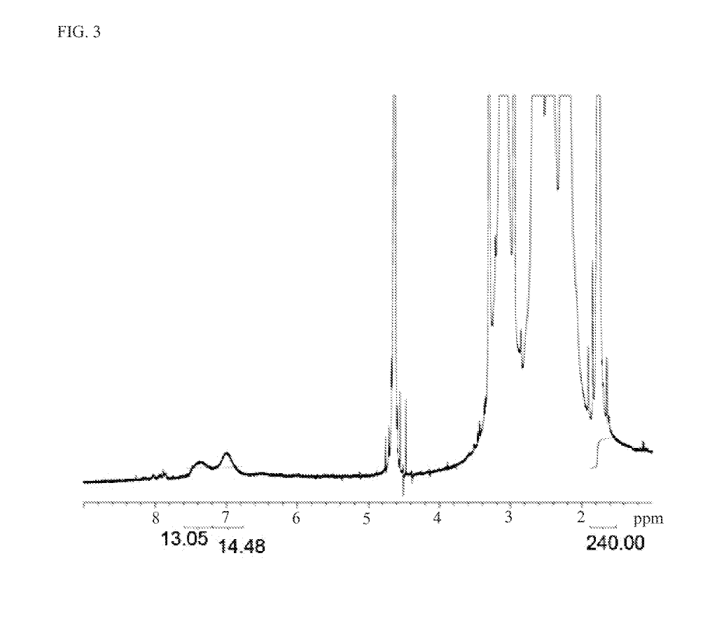

The efficacy of the fluorogenic reporters in monitoring metabolite flux in a typical cell culture system was next evaluated. 5-ethynyl-2′-deoxyuridine (EdU), a nucleoside analogue, incorporation into newly synthesized DNA (Scheme 2), a common molecular biology technique to profile cellular proliferation, was monitored (see, e.g., Hahn, C. G.; Han, L. Y.; Rawson, N. E.; Mirza, N.; Borgmann-Winter, K.; Lenox, R. H.; Arnold, S. E. J Comp Neurol 2005, 483, 154; Salic, A.; Mitchison, T. J. P Natl Acad Sci USA 2008, 105, 2415; Limsirichaikul, S.; Niimi, A.; Fawcett, H.; Lehmann, A.; Yamashita, S.; Ogi, T. Nucleic Acids Res 2009, 37; Yamakoshi, H.; Dodo, K.; Okada, M.; Ando, J.; Palonpon, A.; Fujita, K.; Kawata, S.; Sodeoka, M. J Am Chem Soc 2011, 133, 6102). Traditionally, 5-bromo-2′-deoxyuridine (BrdU), an antibody-specific nucleoside analogue, has been used to monitor DNA synthesis but is limited by the complicated processing and signal variability (see, e.g., Declercq, E.; Descamps, J.; Balzarini, J.; Giziewicz, J.; Barr, P. J.; Robins, M. J. J Med Chem 1983, 26, 661). EdU is a DNA base modified with a terminal alkyne group replacing the methyl group in the 5 position and is readily incorporated into newly synthesized DNA during cell proliferation. The incorporated EdU is readily detected by the ligation of azide fluorescent reporters with the terminal alkyne moiety and has been shown to be a powerful alternative to BrdU for monitoring cell proliferation (see, e.g., Hahn, C. G.; Han, L. Y.; Rawson, N. E.; Mirza, N.; Borgmann-Winter, K.; Lenox, R. H.; Arnold, S. E. J Comp Neurol 2005, 483, 154; Yamakoshi, H.; Dodo, K.; Okada, M.; Ando, J.; Palonpon, A.; Fujita, K.; Kawata, S.; Sodeoka, M. J Am Chem Soc 2011, 133, 6102). To compare the detection efficacy of the different fluorogenic reporters, the DNA-incorporated EdU molecules were ligated with either the small molecule 3-azido-7-hydroxy coumarin or the dendrimer-coumarin reporters (G5-Coumarin 3) using ‘click’ reactions and then analyzed by flow cytometry. KB cells, an epithelial cancer cell line, were incubated with 10 μM EdU for 5 hours to allow incorporation of EdU molecules into newly synthesized DNA. After the cells incorporated EdU molecules into their DNA, the cells were washed and processed allowing for downstream detection of EdU incorporation using click chemistry (see, e.g., Salic, A.; Mitchison, T. J. P Natl Acad Sci USA 2008, 105, 2415). As expected, DNA-incorporated EdU ligation with both fluorogenic reporters (at 10 nM and 100 nM) led to increases in fluorescent intensity (FIG. 2). A much higher degree of cell positive rate was observed with small molecule reporter. However, after standard wash steps, the fluorescence intensity of the small molecule coumarin reporter cells decreased and the cell positive rate returned to the level of dendrimer reporter and Alexa-Fluor 647 azide (AF-647) suggesting that much of the fluorescence signal was due to background fluorescence of the unreacted fluorogenic reporters. AF-647, a standard click-fluorescent reporter, served as the gold standard with its positive signal reflecting the actual amount of EdU incorporation into newly synthesized DNA. In contrast, both concentrations of the dendrimer-reporter (G5-Coumarin 3) and the fluorescent intensity did not change with subsequent wash steps (FIG. 2) demonstrating the signal fidelity of the dendrimer fluorogenic reporter over a broad range of concentrations. KB cells without DNA incorporated EdU did not demonstrate any signal enhancement when reacted with the dendrimer reporter while reaction with the small molecule coumarin reporter led to increases in fluorescence. Similar to other ‘click’ reaction detection methods with traditional azide fluorescent reporters, detection of EdU incorporation into DNA with the fluorogenic dendrimer reporters retain the accuracy of the traditional assays but reduces background fluorescence greatly improving assay throughput.

General Materials and Methods 1H NMR spectra were obtained using a Varian Inova 500 MHz. Matrix-assisted laser desorption ionization time-of-flight mass spectra (MALDI-TOF-MS) were recorded on a PE Biosystems Voyager System 6050, using 2,5-dihydroxybenzoic acid (DHB) as the matrix. Electrospray ionization mass spectra (ESI-MS) were recorded using a Micromass Quattro II Electronic HPLC/MS/MS mass spectrometer.

Materials solvents and chemicals were of reagent grade quality, purchased from Sigma-Aldrich (St. Louis, Mo.), and used without further purification unless otherwise noted Thin-layer Chromatography (TLC) and column chromatography were performed with 25 DC-Plastikfolien Kieselgel 60 F254 (Merck), and Baxter silica gel 60 Å (230-400 mesh), respectively. The dendrimer conjugate G5-NHAc80-(NH12)35 2 was synthesized followed published procedure (see, e.g., Zong, H.; Thomas, T. P.; Lee, K. H.; Desai, A. M.; Li, M. H.; Kotlyar, A.; Zhang, Y. H.; Leroueil, P. R.; Gam, J. J.; Holl, M. M. B.; Baker, J. R. Biomacromolecules 2012, 13, 982). The dendrimer conjugate was analyzed by MALDI-TOF, HPLC, and NMR, the methods of which have been previously described (see, e.g., Mullen, D. G.; Fang, M.; Desai, A.; Baker, J. R.; Orr, B. G.; Holl, M. M. B. Acs Nano 2010, 4, 657). The number of ligands that attached to the dendrimer was obtained from the integration of the methyl protons of the terminal acetyl groups to the aromatic protons on the conjugated ligands (e.g., coumarin). The number of acetyl groups per dendrimer was determined by first computing the total number of end groups from the number average molecular weight from gel permeation chromatography (GPC) and potentiometric titration data for G5-NH2(100%) as previously described (see, e.g., Majoros, I. J.; Thomas, T. P.; Mehta, C. B.; Baker, J. R. J Med Chem 2005, 48, 5892). The total number of end groups was applied to the ratio of primary amines to acetyl groups, obtained from the 1H NMR of the partially acetylated dendrimer, to compute the average number of acetyl groups per dendrimer.

Synthesis of 3-azido-7-((4,6-dichloro-1,3,5-triazin-2-yl)oxy)-2H-chromen-2-one (Triazine-coumarin) (1). To a solution of cyanuric chloride (454 mg, 2.46 mmol) in acetone (10 mL) in an ice-water bath was added diisopropylethylamine (DIPEA) (318 mg, 2.46 mmol). 3-azido-7-hydroxy coumarin (250 mg, 1.23 mmol) in acetone (20 mL) was added slowly over 2 hours. The reaction was stirred at room temperature overnight. The solvent was removed under reduced pressure. The residue was dissolved in CH2Cl2, washed with water, dried over Na2SO4, and rotary evaporated. The resulting residue was purified by column chromatography on silica gel (eluent Hexane/CH2Cl2=50/50) to give 1 as a yellow solid (248 mg, 57%): 1H NMR (500 MHz, CDCl3) δ 7.27 (s, 1H), 7.32 (dd, J1=8.0 Hz. J2=2.0 Hz, 1H), 7.37 (t, J=7.5 Hz, 1H), 7.43 (dd, J1=8.0 Hz, J2=1.5 Hz, 1H); 13C NMR (500 MHz, CDCl3) δ 121.0, 122.8, 125.0, 125.3, 125.8, 127.4, 137.5, 142.5, 155.7, 170.5, 173.3; EST-MS m/z 314.9 (M-Cl—) calculated for C12H4ClN6O3 315.0.

Synthesis of G5-Coumarin (3). To a solution of G5-NHAc80-(NH2)35 2 (53.2 mg, 1.8 μmol) in DMSO (1 mL) was added DIPEA (3.7 mg, 29 μmol). Triazine-coumarin 1 (5.0 mg, 14 μmol) in DMSO (0.2 mL) was added. The reaction was stirred at room temperature overnight. Glycidol (100 μl) was added and the reaction mixture was stirred at room temperature for an additional 24 h. Small molecules were eliminated by five aqueous washings, removing excess water by centrifugation through a membrane filter with 10-kDa cutoff. Purification consisted of ten cycles (20 min at 4800 rpm) using PBS (5 cycles) and DI water (5 cycles). The purified dendrimer samples were lyophilized to yield 3 (54.9 mg, 92%) as yellow solids: MALDI-TOF mass 33294; the 1H NMR integration determined the mean number of coumarin per dendrimer is 3.2.

Experimental Procedure of “Click-on and Probe” Reaction G5-coumarin 3 was dissolved in Cu(II) sulfate (10 mol % per COUlnarin, 1 mg/mL H20 solution) solution. 3-(4-(prop-2-yn-1-yloxy)phenyl)propanoic acid (5 per coumarin, 5 mg/mL DMSO solution) were added. The reaction mixture was incubated at room temperature for 12 h before diluted in H2O for fluorescent emission measurement. For 3-azido-7-hydroxycoumarin, it was dissolved in 3-(4-(prop-2-yn-1-yloxy)phenyl)propanoic acid solution first (5 mg/mL DMSO solution) first, then Cu(II) sulfate and sodium ascorbate aq solution were added to the reaction mixture. The excitation and emission spectra were recorded after dilution using H2O. 1H NMR of G5-Coumarin 3 is shown in FIG. 3.

EdU Labeling and Staining of Cultured Cells. KB cells were in RPMI supplemented with 10% bovine calf serum, penicillin, and streptomycin. EdU was added to the culture media at 10 μM for 5 hours. After labeling, cells were washed two times with PBS followed by fixation with paraformaldehyde. After formaldehyde fixation, cells were rinsed once with TBS and then stained by incubating for 45 min with 100 mM Tris, 0.5 mM CuSO4, fluorogenic reporters or Alexa-Flour 647 azide (10 mM stocks in DMSO), and 50 mM ascorbic acid. After staining, the cells were washed several times with TBS with 0.5% Triton X-100. Fluorescence signal intensities from the samples were measured using an Accuri C6 Flow Cytometer (Ann Arbor, Mich.) and data were analyzed using FlowJo 8.7 software (TreeStar).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-37. (canceled)

38. A composition comprising a dendrimer nanoparticle conjugated with a fluorogenic reporter, wherein said fluorogenic reporter is conjugated with an alkyne chemical moiety, wherein said dendrimer nanoparticle is optionally conjugated with one or more additional functional agents independently selected from the group consisting of a therapeutic agent, a targeting agent, an imaging agent, and a trigger agent.

39. The composition of claim 38, wherein said dendrimer nanoparticle is a PAMAM dendrimer, wherein said fluorogenic reporter is conjugated with an azido chemical moiety, wherein said fluorogenic reporter conjugated with an azido chemical moiety is selected from the group consisting of 3-azido coumarin, 9-azido anthracene, and 6-azido napthalimide, wherein said fluorogenic reporter conjugated with an azido chemical moiety exhibits diminished fluorescence while conjugated with said dendrimer nanoparticle.

40. The composition of claim 38, wherein upon interaction with an alkyne functionalized small molecule via a 1,3-dipolar cycloaddition reaction a triazole ring is formed, wherein said formation of said triazole ring results in detectable fluorescence from said ‘click-on’ fluorogenic reporter.

41. The composition of claim 38, wherein said fluorogenic reporter conjugated with an alkyne chemical moiety is selected from the group consisting of 7-ethynyl coumarin, 2-ethynyl benzothiazole, and 6-ethynyl napthalimide.

42. The composition of claim 39, wherein said fluorogenic reporter conjugated with an alkyne chemical moiety exhibits diminished fluorescence while conjugated with said dendrimer nanoparticle.

43. The composition of claim 38, wherein upon interaction with an azido functionalized small molecule via a 1,3-dipolar cycloaddition reaction a triazole ring is formed, wherein said formation of said triazole ring results in detectable fluorescence from said ‘click-on’ fluorogenic reporter.

44. A method of tracking a small molecule within a biological sample, comprising
providing a dendrimer nanoparticle and a functionalized small molecule, wherein either i) said dendrimer nanoparticle is conjugated with an azido based ‘click-on’ fluorogenic reporter and said functionalized small molecule is an alkyne functionalized small molecule, or ii) said dendrimer nanoparticle is conjugated with an alkyne based ‘click-on’ fluorogenic reporter and said functionalized small molecule is an azido functionalized small molecule, and
introducing said dendrimer nanoparticle and functionalized small molecule into a biological sample,
detecting fluorescence from said dendrimer nanoparticle upon interaction between said dendrimer nanoparticle with said functionalized small molecule, wherein said interaction results in formation of a triazole ring via a 1,3-dipolar cycloaddition reaction, wherein said formation of said triazole ring results in detectable fluorescence from said ‘click-on’ fluorogenic reporter, and
tracking said functionalized small molecule via fluorescence from said ‘click-on’ fluorescence reporter.

45. The method of claim 44,
wherein said dendrimer nanoparticle is a PAMAM dendrimer,
wherein said azido based ‘click-on’ fluorogenic reporter is selected from the group consisting of 3-azido coumarin, 9-azido anthracene, and 6-azido napthalimide,
wherein said alkyne based ‘click-on’ fluorogenic reporter is selected from the group consisting of 7-ethynyl coumarin, 2-ethynyl benzothiazole, and 6-ethynyl napthalimide,
wherein said dendrimer nanoparticle is optionally conjugated with one or more additional functional agents independently selected from the group consisting of a therapeutic agent, a targeting agent, an imaging agent, and a trigger agent,
wherein said functionalized small molecule is selected from the group consisting of a protein, a polysaccharide, a carbohydrate, a lipid, a nucleic acid, an oligonucleotide, and a metabolite,
wherein said biological sample is within an in vivo setting, an ex vivo setting, or an in vitro setting.

46. The method of claim 45, wherein said azido based ‘click-on’ fluorogenic reporter exhibits diminished fluorescence while conjugated with said dendrimer nanoparticle, wherein said detectable fluorescence from said formation of said triazole ring is approximately 20 fold higher in comparison to said diminished fluorescence.

47. The method of claim 46, wherein said alkyne based ‘click-on’ fluorogenic reporter exhibits diminished fluorescence while conjugated with said dendrimer nanoparticle.

48. The method of claim 47, wherein said detectable fluorescence from said formation of said triazole ring is approximately 20 fold higher in comparison to said diminished fluorescence.

49. A method of monitoring a biological process within a biological sample, comprising
providing a dendrimer nanoparticle and a functionalized small molecule, wherein either i) said dendrimer nanoparticle is conjugated with an azido based ‘click-on’ fluorogenic reporter and said functionalized small molecule is an alkyne functionalized small molecule, or ii) said dendrimer nanoparticle is conjugated with an alkyne based ‘click-on’ fluorogenic reporter and said functionalized small molecule is an azido functionalized small molecule, and
introducing said dendrimer nanoparticle and functionalized small molecule into a biological sample,
detecting fluorescence from said dendrimer nanoparticle upon interaction between said dendrimer nanoparticle with said functionalized small molecule, wherein said interaction results in formation of a triazole ring via a 1,3-dipolar cycloaddition reaction, wherein said formation of said triazole ring results in detectable fluorescence from said ‘click-on’ fluorogenic reporter,
tracking said functionalized small molecule via fluorescence from said ‘click-on’ fluorescence reporter, and
monitoring a biological process associated with said functionalized small molecule through tracking said detectable fluorescence.

50. The method of claim 49,
wherein said dendrimer nanoparticle is a PAMAM dendrimer,
wherein said azido based ‘click-on’ fluorogenic reporter is selected from the group consisting of 3-azido coumarin, 9-azido anthracene, and 6-azido napthalimide,
wherein said alkyne based ‘click-on’ fluorogenic reporter is selected from the group consisting of 7-ethynyl coumarin, 2-ethynyl benzothiazole, and 6-ethynyl napthalimide,
wherein said dendrimer nanoparticle is optionally conjugated with one or more additional functional agents independently selected from the group consisting of a therapeutic agent, a targeting agent, an imaging agent, and a trigger agent,
wherein said functionalized small molecule is selected from the group consisting of a protein, a polysaccharide, a carbohydrate, a lipid, a nucleic acid, an oligonucleotide, and a metabolite.

51. The method of claim 50, wherein said azido based ‘click-on’ fluorogenic reporter exhibits diminished fluorescence while conjugated with said dendrimer nanoparticle, wherein said detectable fluorescence from said formation of said triazole ring is approximately 20 fold higher in comparison to said diminished fluorescence.

52. The method of claim 51, wherein said alkyne based ‘click-on’ fluorogenic reporter exhibits diminished fluorescence while conjugated with said dendrimer nanoparticle.

53. The method of claim 52, wherein said detectable fluorescence from said formation of said triazole ring is approximately 20 fold higher in comparison to said diminished fluorescence.

54. The method of claim 50, wherein said biological sample is within an in vivo setting, an ex vivo setting, or an in vitro setting.

55. The method of claim 50, wherein said biological process is selected from the group consisting of nucleic acid related processes, cellular proliferation processes, drug metabolism processes, gene expression processes, protein modification processes, protein interaction processes, physiological processes, reproductive processes, digestion related processes, fermentation related processes, fertilization related processes, germination related processes, tropism related processes, hybridization related processes, metamorphosis related processes, photosynthesis related processes, and transpiration related processes.


SEQUENCE LISTING (1 sequence)

SEQ ID NO: 1 - protein, 12 aa, Artificial Sequence
  Synthetic
HLNILSTLWKYR